(19) United States
(12) Patent Application Publication
(10) Pub. No.: US 2005/0244959 A1
(43) Pub. Date: Nov. 3, 2005

(54) Methods for inducing active hepatocytes proliferation, kits using such methods and uses of said kits

(76) Inventors: Anne Corlu, Rennes (FR); Anne-Laure Serandour, Rennes (FR); Christiane Guguen-Guillouzo, Rennes (FR); Pascal Loyer, Rennes (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/093,016

(22) Filed: Mar. 30, 2005

Related U.S. Application Data
(60) Provisional application No. 60/557,375, filed on Mar. 30, 2004.

(51) Int. Cl. C12N 5/06; C12N 5/08

(52) U.S. Cl. 435/353; 435/370

(57) ABSTRACT

A method for inducing hepatocyte proliferation in long term primary culture involves the steps of: (a) treating the culture with a cytokine and a growth factor; (b) terminating the treatment of step (a) to establish a quiescent phase; and (c) repeating steps (a) and (b) successively to induce several waves of proliferation.

### CROSS-REFERENCE TO RELATED APPLICATION

This application claims priority under 35 U.S.C. § 119 to U.S. Provisional Patent Application Ser. No. 60/557,375, filed Mar. 30, 2004.

The present invention relates to methods for inducing hepatocytes active proliferation. It also relates to screening kits implementing such methods and to specific uses thereof.

### BACKGROUND OF THE INVENTION

In normal liver, hepatocytes are quiescent and highly differentiated. Nevertheless, they have the unique capacity to proliferate after tissue loss, following acute chemical injury or partial hepatectomy (PH). The initial liver mass is restored in a few days by a compensatory growth process, but the anatomical form is not reconstituted. Liver regeneration is mainly dependent on hepatocyte proliferation even if all the other cell types divide to reconstitute the organ specific-lobular-architecture. After 2/3 PH, most hepatocytes proliferate but this peak of activity does not extend beyond one or two cycles, whereas in case of altered hepatocyte proliferation, resident progenitors as well as bone marrow stem cells can contribute to liver repopulation.

Following PH, the complex regenerating process was divided in 3 distinct phases: an initiation step, a proliferation step and a termination step. The initiation phase is characterized by the priming of quiescent hepatocytes and is controlled by pro-inflammatory cytokines, such as TNFα (tumor necrosis factor alpha) and IL-6 (interleukin 6). This process results in inducing hepatocytes to become sensitive to growth factors and competent for replication. However the regulating mechanisms which control and coordinate these events are poorly known. Among growth factors, HGF (hepatocyte growth factor), TGFα (transforming growth factor alpha) and EGF (epidermal growth factor) are mostly susceptible to induce hepatocyte mitogen signal at the restriction point located at 2/3 of the G1 phase. They induce key regulators such as cyclin-dependent-kinases (Cdks) which play a critical role onto cell cycle progression. Their activities are regulated by the binding to cyclins and cdk inhibitors at defined steps of the cell cycle.

In parallel, other studies have reported an important extracellular matrix remodeling at the early stages of liver regeneration, which are necessary for hepatocyte proliferation. Indeed, a periportal infusion of collagenase before administration of growth factors in intact liver induces hepatocyte proliferation, suggesting that extracellular matrix (ECM) degradation and loss of cell to cell contacts may play a role in hepatocyte priming. Moreover, matrix degradation allows a rapid release of HGF in the serum.

ECM degradation is regulated by matrix metalloproteinases (MMPs). These proteases are widely expressed by hepatic cell types and hepatocytes secrete mainly pro-MMP-2 and pro-MMP-9. An induction of pro-MMP-2 and pro-MMP-9 before the phase S entry during liver regeneration has been shown. In addition, previous reports indicate that cytokines such as TNFα and/or IL-6 may play a role in the regulation of pro-MMPs after hepatectomy and in mouse primary hepatocyte cultures.

The inventors then questioned how mitogen signal and extracellular matrix degradation are linked for inducing cell cycle re-entry and progression of differentiated adult hepatocytes. For this purpose, a coculture model associating rat hepatocytes with rat biliary epithelial cells (RLEC) was firstly used. In this culture system, heterotypic cell-cell contacts are restored and a spontaneous early production and deposition of extracellular matrix is observed, mimicking the composition of the hepatic extracellular matrix in liver tissue. In this microenvironment, hepatocytes survive several weeks and their liver specific functions are maintained. In addition, they are unable to proliferate under a growth factor stimulation by EGF or HGF, as in liver.

### BRIEF SUMMARY OF THE INVENTION

The inventors' work allowed to demonstrate that induction of mechanisms similar to those implicated in liver regeneration is possible in vitro. These include the reorganization of cellular communications (cell-cell and cell-extracellular matrix), mitogen signal transduction and cell cycle progression. For instance, cytokine such as TNFα is required for inducing differentiated rat hepatocytes to respond to a growth factor such as EGF, and actively proliferate i.e by completion of multiple cell cycle waves.

Furthermore, in appropriate conditions of stimulation, hepatocytes were able to alternate several phases of proliferation and quiescency. Additionally, the inventors showed how the cytokine TNFα and the growth factor EGF may control the hepatocyte cell cycle progression up to efficient mitosis and that proliferation is dependent on a TNFα-controlled extracellular matrix remodeling.

The present invention relates to methods able to induce active proliferation of hepatocytes.

### BRIEF DESCRIPTION OF THE SEVERAL VIEWS OF THE DRAWINGS

This patent or application file contains at least one photograph executed in color. Copies of this patent or patent application publication with color photgraph(s) will be provided by the Office upon request and payment of the necessary fee.

FIGS. 1 and 2, which refer to the effects of TNFα and growth factors on hepatocyte DNA replication in coculture,

FIG. 3, which concerns the observation by time-lapse microcinematography, of mitosis in mononuclear and binuclear hepatocytes,

FIG. 4, which illustrates the increase of hepacyte number and of hepatocyte cord size, after 12 days of stimulation,

FIG. 5, which deals with the influence of stimulation conditions on hepatocyte proliferation capacity,

FIG. 6, which concerns the extracellular matrix remodeling and regulation of pro-MMP9 expression by TNFα,

FIG. 7, which presents an analysis of differentiation protein levels along successive hepatocyte proliferation waves,

FIG. 8, which relates to the expression, maturation and activity of caspase 8, caspase 9 and caspase 3.

FIG. 9, which refers to the effects of TNFα and EGF on hepatocyte DNA replication in human pure culture,

FIG. 10, which refers to expression of cell-cycle proteins and Cdk activities in EGF and TNFα/EGF stimulated cultures,

FIG. 11, which refer to the effects of FCS, TNFα, EGF and Interleukin 6 on hepatocyte DNA replication in coculture,

FIG. 12, which relates to the expression of cell cycle markers at different stage of the cell cycle, and,

FIG. 13, which concerns the immunostaining of γ tubulin in rat hepatocyte at different step of cell cycle.

### DETAILED DESCRIPTION OF THE INVENTION

In a first embodiment, the invention provides a method for inducing rat hepatocyte proliferation in long term primary culture such as coculture wherein said method comprises the following steps:
a) treating the culture with at least a cytokine and a growth factor, preferably TNFα and EGF,
b) stopping the treatment of step a) to establish a quiescent phase, and,
c) repeating steps a) and b) successively to induce several waves of proliferation.

The quiescent phase allows to restore hepatocyte differentiation status.

Preferably, the coculture associates normal rat hepatocytes with rat biliary epithelial cells.

In another embodiment, the method for inducing rat hepatocyte proliferation in pure culture comprises the following steps:
a) treating the pure culture with a growth factor preferably EGF,
b) stopping the treatment of step a) to establish a quiescent phase,
c) treating the culture with at least a cytokine and a growth factor, preferably TNFα and EGF, and,
d) repeating steps b) and c) successively to induce several waves of proliferation.

In a further embodiment, the invention relates to a method for inducing human hepatocyte proliferation in either pure culture or conditions supporting long term differentiated primary cultures (coculture, collagen or matrigel coating systems.) comprising treating the culture of human hepatocytes with at least a cytokine and a growth factor, preferably EGF and TNFα. Preferably, the cytokine and growth factor treatment is followed by a step of stopping the treatment to establish a quiescent phase, the treatment phase and the quiescent phase being successively repeated to induce several waves of proliferation.

The step of stopping the treatment is essential to induce several waves of proliferation.

Advantageously, the duration of the treatment step is approximately 6 to 12 days, while the duration of the without treatment step is approximately 3 to 5 days, a period needed for restoring the hepatocyte differentiation status and extracellular matrix re-deposition.

Preferably, the hepatocytes are cultured in a medium completed with factors favoring hepatocyte survival and differentiation, such as insulin, corticoids and FCS (fetal calf serum).

The present invention further provides screening kits implementing the above mentioned methods. Such kits are particularly suitable for screening hepatocytes mitogenic activity molecules, for testing toxicity of molecules including genotoxicity, on hepatocytes and for screening molecules remodeling extracellular matrix.

The screening kits according to the invention comprise:
quiescent differentiated hepatocyte cultures, said hepatocyte cultures being preferably selected in the group consisting of rat hepatocytes coculture or long term primary culture, rat hepatocyte pure culture or human hepatocyte pure culture or long term differentiated primary culture,
completed basal medium,
media containing cytokine, growth factor and/or cytokine with growth (for example, TNFα, EGF and/or TNFα with EGF),
protocol(s) describing the above mentioned methods.

Additionally, the screening kits may further comprise:
protocol(s) for analyzing at least one hepatocyte protein selected in the groups consisting of caspases ‘family, metalloproteinases’ family, cyclins and cyclin dependent kinases family, for instance cyclin D1, Cdk2, cyclin E, Cdk1, cyclin B, E2F1, PCNA (proliferating cell nuclear antigen), α tubulin, β tubulin and γ tubulin.

Such kits and uses thereof are particularly described in examples 6-8, 11-12.

The present invention also covers a method of in vivo grafting, which comprises the steps of:
preparing hepatocytes stimulated to proliferate, according to the above described methods, and,
injecting said hepatocytes to the spleen of an animal.

Such a method is more precisely described in example 9.

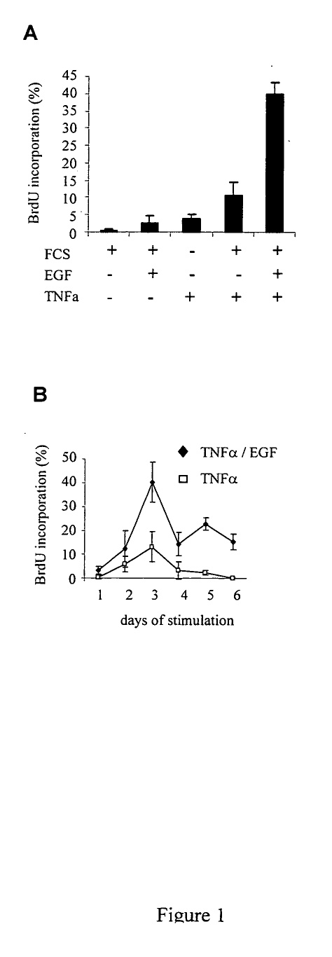

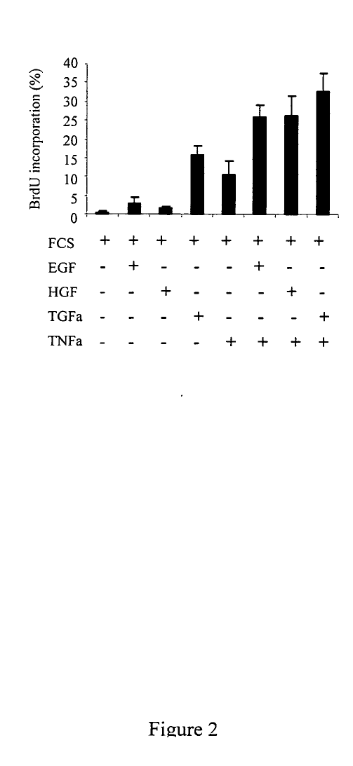

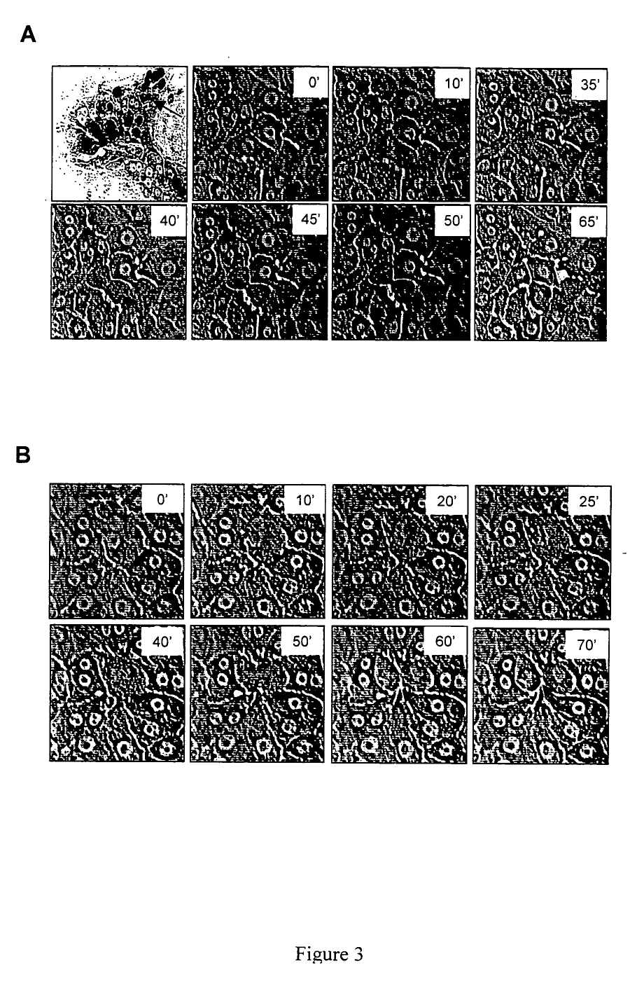

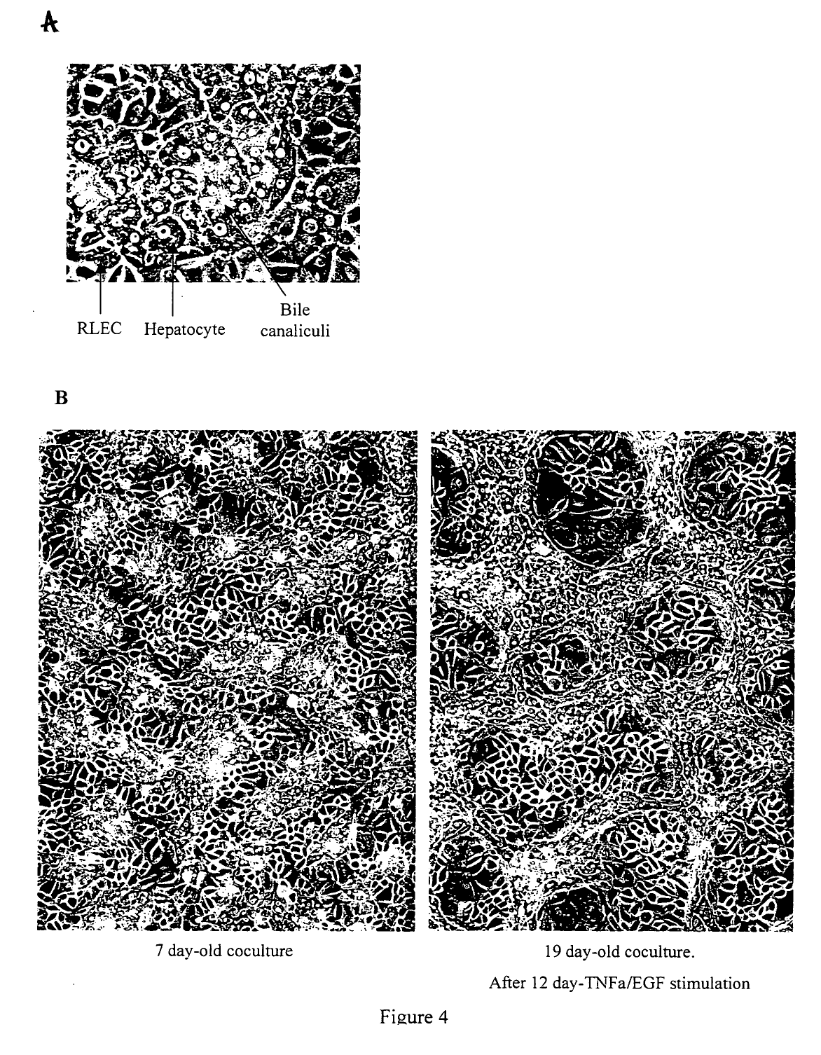

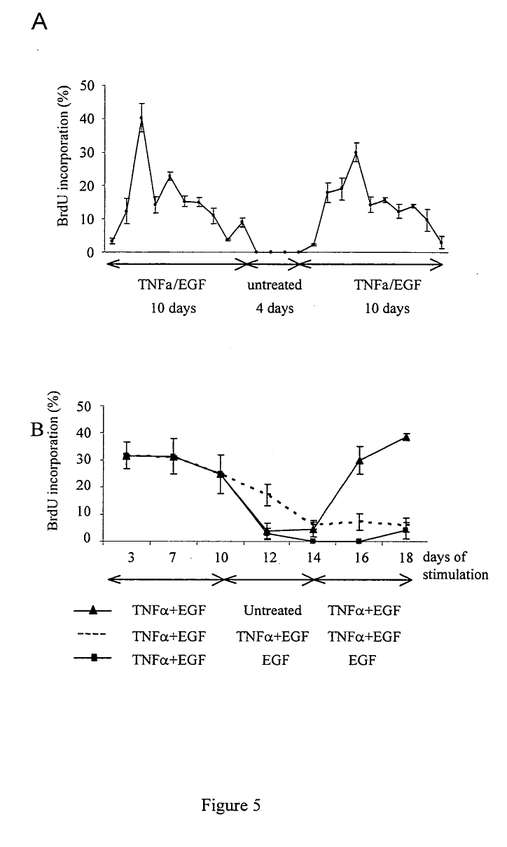

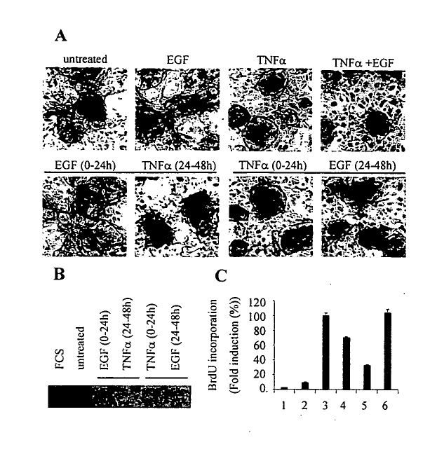

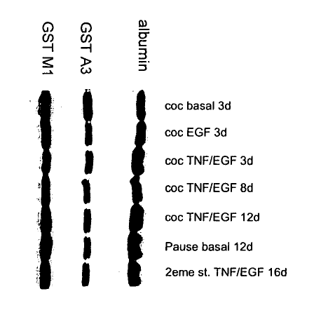

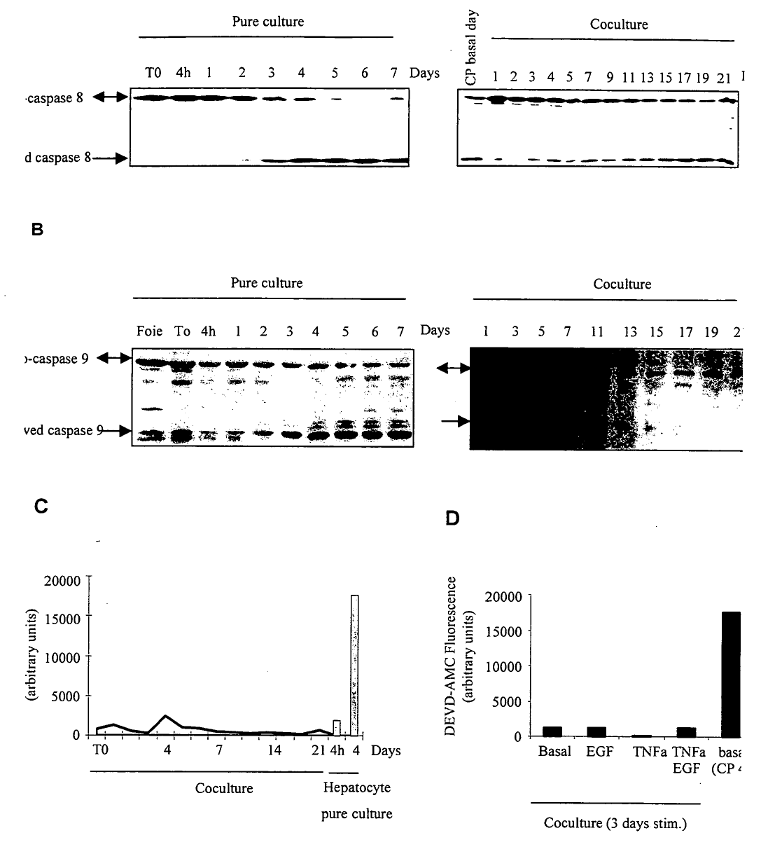

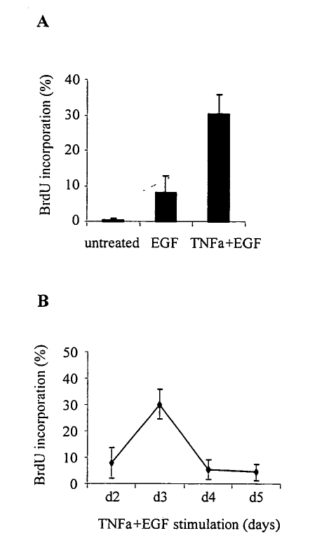

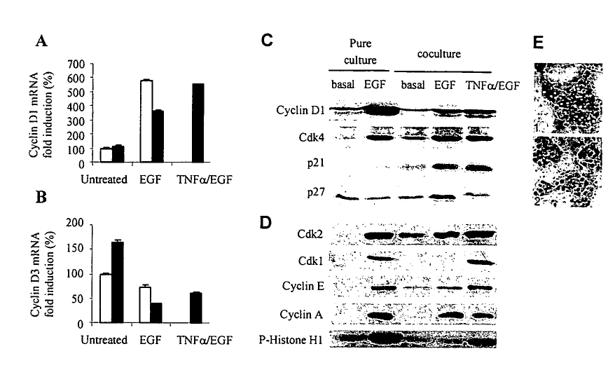

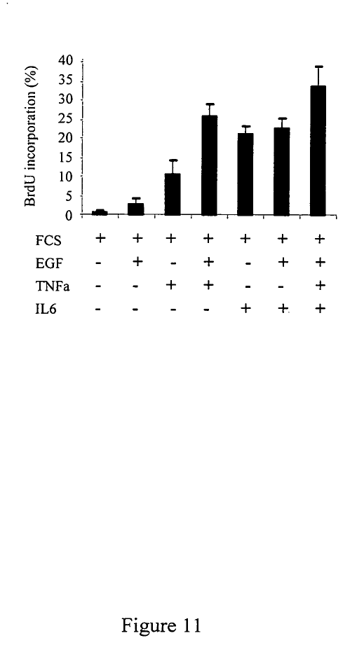

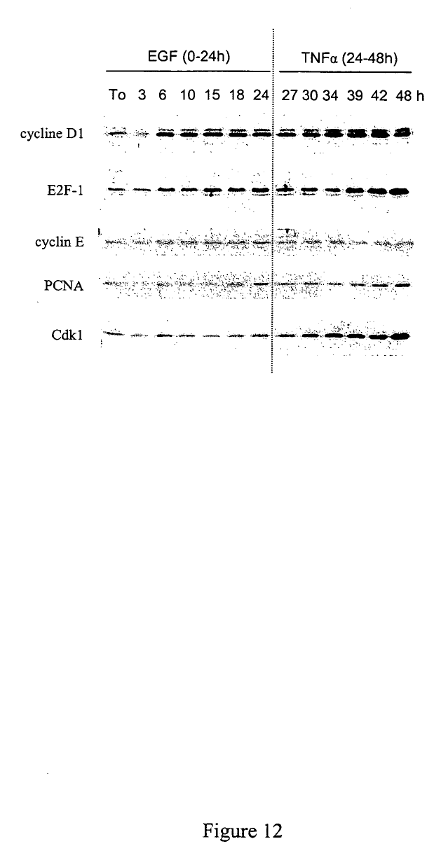

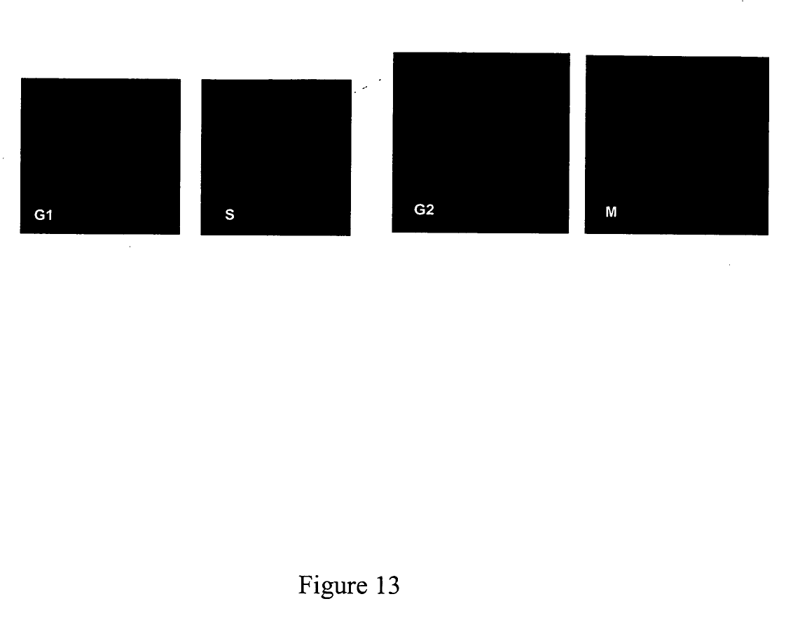

Other advantages and characteristics of the invention will be given in the following examples wherein it will be referred to:
FIGS. 1 and 2, which refer to the effects of TNFα and growth factors on hepatocyte DNA replication in coculture,
FIG. 3, which concerns the observation by time-lapse microcinematography, of mitosis in mononuclear and binuclear hepatocytes,
FIG. 4, which illustrates the increase of hepacyte number and of hepatocyte cord size, after 12 days of stimulation,
FIG. 5, which deals with the influence of stimulation conditions on hepatocyte proliferation capacity,
FIG. 6, which concerns the extracellular matrix remodeling and regulation of pro-MMP9 expression by TNFα,
FIG. 7, which presents an analysis of differentiation protein levels along successive hepatocyte proliferation waves,
FIG. 8, which relates to the expression, maturation and activity of caspase 8, caspase 9 and caspase 3.
FIG. 9, which refers to the effects of TNFα and EGF on hepatocyte DNA replication in human pure culture,
FIG. 10, which refers to expression of cell-cycle proteins and Cdk activities in EGF and TNFα/EGF stimulated cultures,
FIG. 11, which refer to the effects of FCS, TNFα, EGF and Interleukin 6 on hepatocyte DNA replication in coculture,
FIG. 12, which relates to the expression of cell cycle markers at different stage of the cell cycle, and,
FIG. 13, which concerns the immunostaining of γ tubulin in rat hepatocyte at different step of cell cycle.

### EXAMPLES

### Example 1

### Expansion of a Rat Hepatocyte Population by Successive Waves of Proliferation

I. Materials and Methods

Cell Obtaining

Rat Liver Epithelial Cells (RLEC) are originally isolated by trypsinization of 10 day-old rat liver according to the method of Williams et al. (1974, Exp Cell Res, 89:139-42). This cell line is maintained by serial subculture in William's E medium (Eurobio) supplemented with 2 mM L-glutamine (Gibco), 100 μg/ml streptomycin, 100 Ul/ml penicillin (Gibco) and 10% FCS (HyClone). Hepatocytes are isolated from adult male Sprague-Dawley rat (150-200 g) by a two-step collagenase perfusion.

Coculture Protocol (According to Fraslin et al, 1988; Corlu et al, 1991)

Freshly isolated hepatocytes are seeded on plastic dishes at 7×104 cells per cm2 in a mixture of 75% minimal essential medium and 25% 199 medium (Eurobio), supplemented with 2 mM L-glutamine, 0,1% bovine serum albumin (BSA, Sigma), 100 μg/ml streptomycin, 100 Ul/ml penicillin, 5 μg/ml bovine insulin (Sigma), 1.4×10-6 M hydrocortisone hemisuccinate (Roussel) and 10% fetal calf serum (Hyclone). This medium is called “basal medium”.

After cell adhesion, approximately 4 hours after seeding, RLEC, previously trypsinized, are added on spread hepatocytes at 2×105 cells per cm2 in basal medium.

Twenty four hours later, the medium is removed and replaced by a medium supplemented in 7×10-5 M hydrocortisone hemisuccinate, which promotes hepatocyte differentiation and survival. This medium is renewed everyday for 4 days. Then, the concentration of hydrocortisone hemisuccinate is decreased to 7×10-6 M for 2 days.

Cell Proliferation Stimulation

In coculture, hepatocytes restore their differentiated potential within 3-4 days. The inventors have chosen to take well-established coculture at day 7 for demonstrating reversion of quiescent differentiated cells toward cell proliferation activity.

7 day-old cocultures are stimulated with human recombinant EGF (50 ng/ml, Promega) and human recombinant TNF-alpha (10 ng/ml, Promokine) in basal medium for 3 to 10 days. The medium is renewed everyday. As control, cocultures exposed to TNFα alone or EGF are used.

To induce several rounds of hepatocyte proliferation, 7 day-old cocultures are stimulated with human recombinant EGF (50 ng/ml) and human recombinant TNFα (10 ng/ml) in basal medium, for 10 days. Then, a pause in stimulation is performed for 4 days, during which cells are maintained in medium supplemented in 7×10-6 M hydrocortisone hemisuccinate. Next, cocultures are treated with EGF and TNFα in basal medium for 10 days.

To obtain a third wave of proliferation, a pause for 4 days is performed again before TNFα/EGF stimulation.

DNA Synthesis Evaluation

Hepatocyte DNA synthesis is measured by using BrdU (bromodeoxyuridine) labeling. BrdU incorporation in DNA is detected by immunohistochemistry using the Cell Proliferation Kit (Amersham). The number of labeled hepatocytes is determined (5 fields per dish) in order to calculate the percentage of BrdU incorporation.

II. Results

DNA Synthesis of Hepatocytes

7 day-old cocultures are exposed to EGF and/or TNFα for 4 days in a medium supplemented or not with FCS. As shown in FIG. 1A, in untreated cocultures, no BrdU labeling is detected in hepatocytes. In EGF-treated cocultures, as well in TNFα alone, only 3-4% cells are labeled. In contrast, TNFα/EGF induces DNA synthesis in 40% of hepatocytes at day 3 and in 25% at day 4, while less than 10% hepatocytes incorporate BrdU in presence of TNFα/FCS (FIG. 1B). Moreover, several mitotic figures are observed after BrdU labeling in TNFα/EGF stimulation.

When EGF is replaced by HGF (25 ng/ml), similar results are obtained and with TGFα (20 ng/ml), a highest DNA synthesis is observed (FIG. 2).

Hepatocyte Mitosis

7 day-old cocultures stimulated with TNFα/EGF for 4 days are observed by time-lapse micro-cinematography to visualize cytokinesis (FIG. 3). The percentage of dividing cells correlates with the percentage of BrdU labeled cells. Mitosis occurs between 60 and 72 hours of treatment. Secondly, both mononuclear and binuclear hepatocytes, dispersed in colonies, undergo mitosis. In binuclear cells, the two nuclei merged before the prophase and two mononuclear daughter cells are obtained at the cytokinesis (FIG. 3B). Finally, after 12 days of stimulation, an increase of hepatocyte number and of hepatocyte cord size was visualized (FIG. 4).

Obtaining of Several Successive Division Waves

As shown in FIG. 5A, during the first TNFα/EGF treatment, DNA synthesis reaches a maximum at day 3 with 40% of labeled cells, and then decreases slowly. No BrdU incorporation is observed during the unstimulated period, while with the second stimulation, DNA replication starts again with a second peak at the fourth day of TNFα/EGF exposure, before to stop progressively. Cumulative BrdU incorporation during the first and the second stimulation periods reaches 145% of replicating cells. A third wave of proliferation can be obtained following an unstimulated period. Direct role of TNFα in the proliferation signal is ascertained by evidencing NFkB pathway activation.

Requirement of a Pause for Inducing Successive Rounds of Proliferation

After a first 10 day-stimulation with TNFα/EGF, cells are either continuously maintained in TNFα/EGF medium, immediately exposed to EGF alone, kept without any factor for 4 days before a second TNFα/EGF stimulation, or maintained in EGF medium for 4 days before a second TNFα/EGF stimulation (FIG. 5B). Under permanent TNFα/EGF stimulation, as well as with permanent EGF exposure, BrdU incorporation gradually decreases from day 10. Induction of a second wave of DNA synthesis, very similar in magnitude to the first one, is only obtained when a pause in basal medium is performed before a second stimulation. A pause in EGF medium allows DNA synthesis during the second TNFα/EGF stimulation but at a lower level.

This procedure of proliferation induction can be performed in all hepatocyte culture conditions which allow hepatocyte differentiation and cell cycle arrest such as collagen sandwiches, cocultures with endothelial or hematopoietic stromal/mesenchymal cells, liver slices, hepatic micro-bioreactor . . .

### Example 2

### Requirement of an Extracellular Matrix Remodeling for Hepatocyte Proliferation

I. Materials and Methods

Cell Culture

Cell obtaining, coculture initiation and cell proliferation stimulation are performed as described in the example 1.

Extracellular Matrix Deposition Analysis

Matrix fibers are visualized in cocultures fixed with a mixture of 4% paraformaldehyde and 2.5% glutaraldehyde in 0.1 M cacodylate buffer (pH 7.4) for 15 min at 4° C. Then the reticulin staining by silver impregnation of extracellular matrix is carried out according to the method of Gordon and Sweets and applied to coculture (Exp Cell Res 1984; J Cell Biol 1991). Matrix components are analysed by immunostaining using antibodies against fibronectin, collagen I, collagen III)

Gelatin Zymography

Destruction or remodeling of the extracellular matrix are associated with activation of specific proteinase enzyme.

7 day-old cocultures are stimulated with different combinations of TNFα and/or EGF in a FCS-free medium for 24 h. Twenty μl of supernatant are electrophoresed under nonreducing conditions on 12% SDS-polyacrylamide gel containing 0,1% bovine skin gelatin (Sigma) as substrate. After electrophoresis, the gel is washed twice with 2.5% Triton X-100 for 10 min and twice with water for 20 min. After incubation overnight at 37° C. in reaction buffer (50 mM Tris-HCL, pH 8; 5 mM CaCl2; 5 μM ZnCl2), the gel is stained with Coomassie Brillant Blue and destained. Clear zones in blue background indicate the presence of metalloproteinase pro-MMP9 activity; 5% FCS is used as positive control.

Inhibition of Extracellular Matrix Remodeling

7 day-old cocultures are stimulated with EGF and then with TNFα for 24 h periods. Factors are added in a FCS-free medium. Inhibition of extracellular matrix degradation is induced with 1,10-Phenanthroline monohydrate (Sigma), a chelator of metal ions which inhibits the metalloproteinase activity. This compound is soluble in dimethylsulfoxyde and is used at 1 μM.

II. Results

Matrix Extracellular Remodeling During Hepatocyte Proliferation

As shown in FIG. 6A, in both untreated and EGF-treated conditions, extracellular matrix (ECM) is very abundant. Fibers are mainly located within and around hepatocyte colonies.

When cocultures are treated with TNFα, the ECM deposition is very sparse. In TNFα/EGF condition, most fibers are degraded and disappear in proliferating colonies.

Induction of Metalloproteinase MMP9 Expression During Hepatocyte Proliferation

Pro-MMP9 (92 Kda) is evidenced by zymography (FIG. 6B). This enzyme is not detected in untreated and EGF-treated cocultures. Pro-MMP9 is only expressed in TNFα (or TNFα/EGF stimulated cocultures). Its expression parallels disruption of the extracellular matrix fibers.

Inhibition of Both ECM Degradation and Hepatocyte Proliferation by Phenanthroline

As shown in FIG. 6C, the DNA synthesis is strongly decreased when phenanthroline is added to cocultures treated with EGF for 24 h and then with TNFα. In addition, this inhibition is reversed by TNFα after phenanthroline removal and DNA synthesis is completely restored within 24 h. The ECM degradation inhibition by phenanthroline and the remodeling by TNFα are controlled by reticulin staining in all experiments.

### Example 3

### High Differentiation Status of Hepatocyte Population Required for Succeeding Proliferation Stimulation

I. Material and Methods

Cell Culture

Cell obtaining and coculture initiation are performed as described in the example 1.

Coculture Stimulation

Successive waves of hepatocyte proliferation are performed. 7 day-old cocultures are stimulated with human recombinant EGF (50 ng/ml) and human recombinant TNFα (10 ng/ml) in basal medium, for 10 days. Then, a pause in stimulation is performed for 4 days, during which cells are maintained in medium supplemented in 7×10-6 M hydrocortisone hemisuccinate. Next, cocultures are treated with EGF and TNFα in basal medium for 10 days.

Protein Analysis

Only hepatocyte fractions are used. Hepatocytes are selectively separated from RLEC by incubation in a calcium-free HEPES-buffered collagenase B solution (0.08%; pH 7.4) for 30 min at 37° C. Hepatocytes that are more sensitive to low concentration of Ca2+ become rounded and then detach in clumps, whereas RLEC remain well spread.

Hepatocyte fractions are lysed and proteins are electrophoresed on 12% SDS-polyacrylamide gel. Markers such as albumin, transferrin, Glutathion-S-Transferase (GST) are analyzed.

II. Results

Preservation of Morphological Characteristics of Hepatocytes During Proliferation

Time-lapse microcinematography analysis revealed that the cell population preserves most of the differentiated morphological characteristics during proliferation stimulation. For instance, the biliary pole is maintained. However, hepatocyte colonies become flattened in parallel to matrix fiber degradation. At the time of cell progression to mitosis, changes of cell shape are observed but bile canaliculi are seen immediately after cytokinesis and their periodic swelling early propagates along the plasma membrane of daughter cells (FIG. 4A).

Expression of Hepatic Functions

Differentiation protein expression levels are analyzed along successive hepatocyte proliferation waves (FIG. 7). High levels of albumin production as well as Glutathion-S-Transferase A3, M1 isozymes, which are involved in drug metabolism, are expressed at the same level in untreated coculture and along TNFα/EGF treatment. These results indicate that hepatocytes maintain a high level of differentiation until their progression to S phase and DNA synthesis and early restore their functional properties in the daughter cells. From this observation, it is strongly expected that all hepatocyte culture conditions able to preserve a high level of differentiation are relevant for obtaining efficient proliferation stimulation by using the herein protocol.

### Example 4

### Arrest of Apoptosis Progression in the Defined Conditions of Hepatocyte Proliferation

I. Material and Methods

Cell Culture

Cell obtaining, coculture initiation and cell proliferation stimulation are performed as described in the example 1.

Reagents

Anti-caspase 8 (APP-108) and anti-caspase 9 were from StressGen Biotecnologies Corp.

Fluorogenic subtrayes are from BACHEM and prepared at 100 mM in the recommended solvent.

Caspase Activity Assay

Caspases are cysteine-rich proteases which specifically cleave proteins with aspartic residues and this activity can be measured using a test based on the degradation of a modified peptide labeled by a fluorescent molecule. Hepatocytes and liver biopsies are lysed in the caspase activity buffer (Stennicke, H. R., and Salvesen, G. S. (1997) J Biol Chem 272, 25719-25723). 100 μg of crude cell lysate are incubated with 80 μM substrate-AMC for 1 hour at 37° C. Caspase mediated cleavage of peptide-AMC (7-amino 4-methylcoumarin) is measured by spectrofluorimetry (Molecular Devices) at the excitation/emission wavelength pair (ex/em) of 380/440 nm. The caspase activity is given in arbitrary units of fluorescence (per 100 μg of total proteins). DEVD-AMC is the substrate used for caspases 3 and 7 activity measurement.

Immunoblotting Analysis

Cells from total extracts or only hepatocyte fractions were harvested after different stimulation conditions and lysed in a buffer containing 50 mM HEPES (pH 7.5), 150 mM NaCl, 1 mM EDTA, 2.5 mM EGTA, 0,1% Tween 20,1 mM DTT, 0.1 mM sodium orthovanadate, 1 mM NaF, 10 mM β-glycerophosphate, 0.1 mM phenylmethylsulfonyl fluoride, and 100 μg/ml benzamidine and protease inhibitor cocktail (5 μg/ml aprotinin, leupeptin, pepstatin, and soybean trypsin inhibitor) and sonicated in ice. Protein concentrations were determined by Bio-Rad protein assay. Proteins were separated on 12% SDS-polyacrylamide gels and transferred onto nitrocellulose membranes (Schleicher Schulle). The protein amounts in each lane were controlled by staining membranes with Ponceau Red. Primary antibodies were incubated overnight at 4° C. in a 3% nonfat dry milk-TBS-0,1% Tween 20 solution. Then, membranes were washed 3 times in TBS-0,1% Tween 20 solution. Secondary antibodies conjugated to horseradish peroxidase were incubated in a 3% nonfat dry milk-TBS-0,1% Tween 20 solution for 1 hour at room temperature, and then membranes were washed again. Immunoreactive bands were detected using the SuperSignal™ Ultra Chemiluminescent Substrate (Pierce).

II. Results

Maturation of Procaspase 8 But not 9 in Coculture

The inventors have focused their attention onto the expression, maturation and activity of caspase 8 (initiator), caspase 9 (mitochondrial) and caspase 3 (executioner). Indeed, these protease have been found to be activated in hepatocytes maintained in pure culture.

First, the expression and the maturation of these caspases have been studied by western-blotting from 7 day-old cocultures stimulated or not by TNFα/EGF and harvested at different times for 10 days. Procaspase 8, present in normal liver, is highly expressed in freshly isolated hepatocytes, strongly decreases within 3 days in pure culture while the cleaved form accumulates (FIG. 8A). In unstimulated coculture, this cleaved form appears at day 3, gradually accumulates and becomes clearly expressed in 7 day old coculture at the time of proliferation stimulation. However, even in presence of this cleaved from, no caspase 8 activity can be detected. In addition, in contrast to pure culture, no caspase 9 maturation can be seen in coculture (FIG. 8B).

Secondly, executioner caspase 3/7 activity has been measured. In coculture, the level of its activity is very low compared to that observed in pure culture and this strongly indicates a blockage of apoptosis in coculture (FIG. 8C). In order to induce hepatocyte proliferation wave, TNFα/EGF is added to 7 day-old coculture. As TNFα is an efficient apoptotic cytokine, whether exposure to this cytokine could induce apoptosis was sought. As shown on FIG. 8D, caspase 3/7 activity remains very low all along stimulation, indicating that coculture conditions preserve from apoptosis presumably by the presence of cell-cell communication mediated survival signal, that makes possible rapid reversion toward differentiation status.

### Example 5

### Induction of Adult Human Hepatocyte Proliferation in Pure Culture

I. Material and Methods

Cell Obtaining

Non tumoral part of patient liver resection samples are dissociated using enzymatic procedure. Briefly, normal hepatocytes are isolated by a two-step collagenase perfusion as described by Guguen-Guillouzo et al (1982, Cell Biol Int Rep, 6:625-628).

Pure Culture of Human Hepatocytes

Freshly isolated hepatocytes are seeded on plastic dishes at 5×104 cells per cm2 in a William's E medium (Eurobio) supplemented with 2 mM L-glutamine (Gibco), 0,1% bovine serum albumin (BSA, Sigma), 100 μg/ml streptomycin, 100 Ul/ml penicillin (Eurobio), 50 μg/ml gentamycine (Sigma), 5 μg/ml bovine insulin (Sigma), and 10% fetal calf serum (HyClone).

Proliferation of Human Hepatocytes

Twenty four hours after seeding, human hepatocytes are stimulated with recombinant human TNFα (Promokine, 10 ng/ml) and recombinant human EGF (Promega, 50 ng/ml). These factors are added in William's E medium (Eurobio) supplemented with 2 mM L-glutamine (Gibco), 0,1% bovine serum albumin (BSA, Sigma), 100 μg/ml streptomycin, 100 Ul/ml penicillin (Eurobio), 50 μg/ml gentamycine (Sigma), 5 μg/ml bovine insulin (Sigma), 1.4×10-6 M hydrocortisone hemisuccinate (Roussel), 10% fetal calf serum. This medium is renewed every day and stimulation lasts at least 2 days.

DNA Synthesis Measurement

BrdU is incubated in medium during the last 24 h of treatment. Then pure cultures are fixed with a mixture of 90% ethanol, 5% acid acetic and 5% water for 20 min at 4° C. BrdU incorporation in DNA is detected by immunohistochemistry using the Cell Proliferation kit (Amersham). Next, cytoplasm and nuclei are stained in blue with Hemalun (Merck). The numbers of labeled hepatocytes are determined (5 fields per dish) in order to calculate percentage of BrdU incorporation.

II. Results

Human Hepatocyte Proliferation

Twenty four hours after seeding, pure cultures of human hepatocytes are stimulated with EGF alone or TNFα/EGF for 48 h. The combination TNFα/EGF induces DNA synthesis in up to 30% of hepatocytes whereas EGF alone only induces 8% of cells to enter in S phase (FIG. 9A). Similar results are obtained with other growth factors such as HGF or TGFα.

Time course of TNFα/EGF stimulation shows a transient peak of replication at day 3 and then the BrdU incorporation level strongly decreases (FIG. 9B).

### Example 6

### Use of the Hepatocyte Proliferation Procedure to Screen Chemicals or Drug Molecules Susceptible to Induce or Favor Mitogenic Activity

A Test is Proposed for

1) eliciting new therapeutic targeting for acute hepatitis diseases and liver regeneration activity. Inducers of proliferation can be detected as described in example 1.

2) detecting chemicals able to induce abnormal hepatocyte proliferation in the absence of tissue loss and resulting in apoptotic compensatory reaction in order to restore liver homeostasis. Such chemicals are potential inducers of cancerous cell occurrence.

The Kit Includes:

Settled quiescent differentiated hepatocyte cultures of human or other species origin in multiwell dishes,
Complete basal medium ready for use,
Media containing TNFα or EGF alone or TNFα/EGF
A protocol for stimulation of one or two waves of proliferation, a protocol for BrdU staining and a protocol of caspase activity assays.
   Description of the Test:

One Wave of Proliferation

Molecules are Tested in Three Different Media:

1) basal medium
2) basal medium supplemented with human recombinant EGF to identify molecules which can induce hepatocyte priming, like pro-inflammatory molecules;
3) basal medium supplemented with human recombinant TNFα to identify molecule with growth factor activity.

Treatments are performed for 3 days. The medium is renewed everyday.

As positive controls, cultures are stimulated with human recombinant EGF and human recombinant TNFα in basal medium for 3 days. The medium is renewed everyday.

As negative controls, cultures are maintained in basal medium for 3 days. The medium is renewed everyday.

Proliferation analysis is performed between day 2 and 3 with Brdu incorporation. Apoptosis can be evaluated by caspase 8 and caspase 3 activity assays

Successive Waves of Proliferation

To get conditions for testing successive waves of proliferation, cells are treated first for 8 days with the molecule in basal medium supplemented or not with EGF or TNFα. Then, cells are maintained in basal medium for 4 days before the second stimulation in basal medium with the molecule and supplemented or not with EGF or TNFα. In parallel, to verify cell proliferation ability, wells are treated with EGF and TNFα for 8 days, without cytokine for 4 days and then with EGF and TNFα for 8 additional days.

Proliferation analysis is performed by 24 h BrdU incorporation. Apoptosis can be evaluated at different times of the culture by caspase8 and caspase3 activity assays.

### Example 7

### Use of Proliferating Hepatocytes from Human or Rodent Origins to Screen Toxicity of Chemicals

Toxicity mechanisms which can be analyzed in proliferating culture are:
1) Induction of apoptotic pathways. Direct benefit results from that in normal proliferating coculture, apoptosis is low. Caspase 9, 8, 3 activities can be analyzed as described in example 4.
2) Blockage of hepatocyte proliferation in the cell cycle (late G1, phases S or M). It can be evaluated as described for phenanthroline and IFNγ in example 2 or for iron chelators by Rakba et al., Carcinogenesis. 2000 May 21 (5):943-51. Progression in cell cycle can be defined by expression of cell cycle markers. The progression of proliferating hepatocytes in cell cycle after TNFα/EGF stimulation is analyzed in FIG. 10,
3) Production of reactive metabolites (free radicals, DNA adducts . . . ), mainly occurring in dividing cells.
4) Carcinogen molecules most active during DNA replication.
   The Kit Includes:
Settled quiescent differentiated hepatocyte cultures of human or other species origin in multiwell dishes,
Complete basal medium ready for use,
Media containing TNFα or EGF alone or TNFα/EGF
A protocol for stimulation of one wave of proliferation, a protocol for BrdU staining and a protocol of caspase activity assays.
   Description of the Test:

Molecules are tested in medium containing EGF and TNFα. Treatments are performed for 3 days. The medium is renewed everyday. As positive controls, cultures are stimulated with human recombinant EGF and human recombinant TNFα in basal medium for 3 days. The medium is renewed everyday.

As negative controls, cultures are maintained in basal medium for 3 days. The medium is renewed everyday.

Proliferation analysis is performed between day 2 and 3 with Brdu incorporation. Apoptosis can be evaluated at different times of the culture by caspase8 and caspase3 activity assays. Cell cycle and/or specific hepatic protein expression can be analyzed by western blotting.

### Example 8

### Use of Hepatocyte-Associated Extracellular Matrix Deposition to Evaluate the Activity of Molecules Susceptible to Remodeling Extracellular Matrix and Useful for Hepatic Fibrosis Reversion

The principle is based on the observation in vivo that hepatocyte regeneration is inhibited in patients suffering from fibrosis and cirrhosis. The use of long term primary cultures of human hepatocytes and/or cocultures in which extracellular matrix fibers accumulate, would allow to test new therapeutics able to degrade these fibers while preserving hepatocyte survival potencies and proliferation activity. Interleukin 1 and 6 are known to modulate extracellular matrix remodeling. In coculture, the inventors have shown that interleukin 6 at 5 ng/ml allow hepatocytes to respond to growth factors (FIG. 11).

The Kit Includes:

Settled quiescent differentiated hepatocyte cultures of human or other species origin in multiwell dishes,
Complete basal medium ready for use,
Media containing TNFα or EGF alone or TNFα/EGF
A protocol for stimulation of one or two waves of proliferation, a protocol for BrdU staining, a protocol for reticulin staining and a protocols to study metalloproteinase activities.
   Description of the Test:
   Molecules are Tested in Different Media in Absence of Fetal Calf Serum:
1) medium supplemented with recombinant EGF to identify molecules which can induce degradation of extracellular matrix like MMPs.
2) medium supplemented with human recombinant TNFα and EGF to identify molecule which can induce degradation of extracellular matrix like MMPs and/or modulate MMP inhibitor.

Treatments are performed for 24 hours or 3 days. In the last case, the medium is renewed everyday.

As positive controls, cultures can be stimulated for 24 hours with EGF and then 24 hours with TNFα or stimulated with EGF and TNFα for 3 days. The medium is renewed everyday.

As negative controls, cultures are maintained in basal medium for 24 hours or 3 days. The medium is renewed everyday.

Proliferation analysis is performed with Brdu incorporation during the first 24 hours of treatment or between day 2 and 3. Extracellular matrix deposition or Metalloproteinase expression are analyzed after 24 h or 3 days of stimulation.

### Example 9

### Use of Proliferating Hepatocytes for In Vivo Grafting and Repopulation of Damaged Liver

Proliferating normal hepatocytes could come from rodents or mainly from human origins. It has been well demonstrated that injection of suspension of adult human hepatocytes in the spleen of immunodeficient mouse allows migration to the liver and formation of human hepatocyte nodules distributed into the mouse parenchymal tissue. Percentage of repopulation is greatly increased by providing a selective advantage to the grafting cells by provoking destruction of the liver using spatio-temporal controlled induction of the urokinase/plasminogen activation (UPA) gene leading to matrix degradation and massive cell death (transgenic UPA immunodeficient mice). However, the percentage of repopulation in these animals with human hepatocytes remains lower than 15-20% and cells aggregate in nodules which are rapidly surrounded with fibrotic fiber deposition that strongly limits exchanges with the neighboring tissue limiting permissivity to therapeutics or infectious agents instead of quiescent cells.

The goal is to improve repopulation and tissue reorganization by using proliferating hepatocytes.

I. Materials and Methods

Pure culture of human hepatocytes stimulated to proliferate by 2 day exposure to TNFα/EGF will be injected to the spleen of Rag2−/− UPA transgenic mice according to Dandry et al, 2001, Hepatology, 33; 981-988). Repopulation of mouse liver with proliferating or non proliferating human hepatocytes will be compared by measuring human plasmatic proteins secreted into the blood (Elisa technics).

This in vitro model allows not only to screen toxicity of chemicals in human hepatocytes in vivo but also to study infection by human hepatic specific parasites, mainly viruses, and to test efficiency of new therapeutic drugs.

### Example 10

### Adapted Protocol for Specific Hepatocyte Gene Transfection

Adult hepatocytes are poorly permissive to gene transfection. This low efficiency is mainly due to the non-proliferative status of the cells. Another causal effect would be related to the apoptotic pathway inducted in cells because of inappropriate culture conditions.

Use of proliferating hepatocytes prepared according to the protocol adapted to minimize cell apoptotic progression allows to:
improve efficiency of viral vectors (mainly retroviruses) and non-viral synthetic vectors.
favor cell survival following transfection process.
Applications include academic research but also cell and gene therapy in human.

### Example 11

### Use of the Hepatocyte Proliferation Procedure to Evaluate the Molecular Mechanisms of Drugs Susceptible to Induce or Inhibit Cell Cycle

A test is provided to analyze the mechanism of cell cycle blockage or progression (early G1, Late G1, S and M) induced by drugs by studying the expression of cell cycle markers which sign different steps of cell cycle (cyclin D1, E2 F1, Cdk2, cyclin E, Cdk1, cyclin B, PCNA, alpha tubulin). Successive 24 h treatments of cocultures by molecules added alone or in combination with EGF or TNFα are used to determine the effect of each molecule. In coculture, the inventors show that EGF promotes hepatocyte cell cycle progression to late G1 by inducing expression of cyclin D1, EF21 and cyclin E, while TNFα allows S entry through Cdk1 induction (FIG. 12).

The Kit Includes:

Settled quiescent differentiated hepatocyte cultures of human or other species origin in multiwell dishes,
Complete basal medium ready for use,
Media containing TNFα or EGF alone or TNFα/EGF
A protocol for stimulation of one wave of proliferation, a protocol for BrdU staining and a protocol of cell cycle protein analysis.
   Description of the Test:

Step 1: Identification of the Molecule Effect by DNA Synthesis Measurement

Molecules are tested in medium containing TNFα or EGF alone or TNFα/EGF. Treatments are performed for 3 days. The medium is renewed everyday.

As positive control, cultures are stimulated with TNFα and EGF in basal medium for 3 days. The medium is renewed everyday.

As negative control, cultures are maintained in basal medium for 3 days. The medium is renewed everyday.

Proliferation analysis is performed between day 2 and 3 with BrdU incorporation. Inhibitory and activating molecules are discriminated.

Step 2: Cell Cycle Analysis

Cells are seeded in duplicate and are successively treated for 24 hour periods as described hereunder

| 0-24 hours | 24-48 hours | |
|---|---|---|
| basal | basal | Negative control |
| EGF | EGF | Late G1 control |
| EGF | TNFα | Positive control |
| X | TNFα | |
| EGF + X | TNFα | |
| EGF | X | |
| EGF | X + TNFα | |

X: tested molecules

Cells are arrested at each time (24 and 48 h) and protein extracts are prepared and separated in SDS-polyacrylamide gel. Cell cycle markers are analyzed by western blot using corresponding antibodies.

### Example 12

### Use of the Hepatocyte Proliferation Procedure to Evaluate Molecules Susceptible to Block Cell Growth by Interfering in Mitotic Spindle Formation

To proliferate successfully, cells need to coordinate both the centrosome duplication and segregation with the chromosome separation. Aberration in the centrosome cycle and mitotic spindle formation is implicated in aneuploïdy and cancer development. The hepatocyte proliferation procedure allows screening of molecules susceptible to block centrosome cycle and cell division. (See FIG. 13)

The Kit Includes:

Settled quiescent differentiated hepatocyte cultures of human or other species origin in multiwell dishes,
Complete basal medium ready for use,
Media containing TNFα and EGF
A protocol for stimulation of one wave of proliferation, a protocol for BrdU staining and a protocol of γ tubulin (a centrosomal protein) immunofluorescence.
   Description of the Test:

Molecules are tested in duplicate in medium containing TNFα and EGF. Treatments are performed for 3 days. The medium is renewed everyday.

As positive control, cultures are stimulated with human recombinant EGF and human recombinant TNFα in basal medium for 3 days. The medium is renewed everyday.

As negative control, cultures are maintained in basal medium for 3 days. The medium is renewed everyday.

Proliferation analysis is performed between day 2 and 3 with BrdU incorporation.

In parallel, cells are fixed and γ tubulin is detected by immunofluorescence. Number and localization of centrosome in hepatocyte is determined.

1. Method for inducing rat hepatocyte proliferation in long term primary culture wherein said method comprises the following steps:
a) treating the culture with at least a cytokine and a growth factor,
b) stopping the treatment of step a) to establish a quiescent phase, and,
c) repeating steps a) and b) successively to induce several waves of proliferation.

2. Method according to claim 1, wherein the long term primary culture is a coculture associating normal rat hepatocytes with rat biliary epithelial cells.

3. Method for inducing rat hepatocyte proliferation in pure culture comprises the following steps:
a) treating the pure culture with a growth factor,
b) stopping the treatment of step a) to establish a quiescent phase,
c) treating the culture with at least a cytokine and a growth factor, and,
d) repeating steps b) and c) successively to induce several waves of proliferation.

4. Method for inducing human hepatocyte proliferation in either pure culture or conditions supporting long term differentiated primary cultures comprising treating the culture of human hepatocytes with at least a cytokine and a growth factor.

5. Method according to claim 4, wherein the cytokine and growth factor treatment is followed by a step of stopping the treatment to establish a quiescent phase, the treatment phase and the quiescent phase being successively repeated to induce several waves of proliferation.

6. Method according to claims 1, 3 or 4, wherein the duration of the treatment step is approximately 6 to 12 days.

7. Method according to claims 1, 3 or 5 wherein the duration of the without treatment step is approximately 3 to 5 days.

8. Method according to any of the preceding claims wherein the hepatocytes are cultured in a medium completed with factors favoring hepatocyte survival and differentiation.

9. Screening kit comprising:
quiescent differentiated hepatocyte cultures,
completed basal medium,
media containing cytokine, growth factor and/or cytokine with growth,
protocol describing the method according to any of the claims 1 to 8.

10. Screening kit according to claim 9, which further comprises:
protocol(s) for analyzing at least one hepatocyte protein selected in the groups consisting of caspases ‘family, metalloproteinases’ family, cyclins and cyclin dependent kinases family, for instance cyclin D1, Cdk2, cyclin E, Cdk1, cyclin B, E2 F1PCNA (proliferating cell nuclear antigen), α tubulin, β tubulin and β tubulin.

11. Use of the kit according to claim 9 or 10 for screening mitogenic activity molecules.

12. Use of the kit according to claim 9 or 10 for screening toxicity of molecules.

13. Use of the kit according to claim 9 or 10 for screening molecules remodeling extracellular matrix.

14. Method of in vivo grafting, which comprises the steps of:
preparing hepatocytes stimulated to proliferate, according to any
method of the claims 1 to 8, and,
injecting said hepatocytes to the spleen of an animal.
